(19) United States
(12) Patent Application Publication
(10) Pub. No.: US 2010/0022576 A1
(43) Pub. Date: Jan. 28, 2010

(54) STABLE AND BIOAVAILABLE FORMULATIONS AND A NOVEL FORM OF DESLORATADINE

(76) Inventors: Ramaswami Bharatrajan, Thane (West) (IN); Kamalakar Talasila, Thane (West) (IN)

Correspondence Address: Manelii Denison & Selter PLLC, 2000 M Street, 7th Floor, Washington DC, DC, 20036, US

(21) Appl. No.: 12/375,038

(22) PCT Filed: Jun. 5, 2007
(86) PCT No.: PCT/EP2007/004980

§ 371 Date: Jan. 26, 2009

(30) Foreign Application Priority Data
Jun. 7, 2006 (IN) 874/MUM/06

(51) Int. Cl. A61K 31/435; A61P 37/08

(52) U.S. Cl. 514/290

(57) ABSTRACT

An active pharmaceutical ingredient comprising 95% of form I of desloratadine and 5% of form II of desloratadine is disclosed. Also disclosed are a process for its preparation as well as a bioavailable and stable formulation containing this active pharmaceutical ingredient for the treatment of allergic diseases like allergic rhinitis, chronic idiopathic urticaria, asthma and other similar diseases. The compositions are formulated such as to provide protection to the drug from acidic excipients. The compositions of the present invention comprise desloratadine intimately mixed with hydrogenated vegetable oil and certain other excipients including acidic excipients.

### CROSS-REFERENCE TO RELATED APPLICATION

This application claims priority to Indian Provisional Application 874/MUM/2006, filed on Jun. 7, 2006, and entitled “stable and bioavailable formulations and a novel form of desloratadine”, the contents of which are incorporated by reference herein.

### FIELD OF THE INVENTION

The present invention pertains to an active pharmaceutical ingredient comprising desloratadine and to stable and bioavailable pharmaceutical compositions comprising desloratadine.

The oral dosage forms of the present invention are useful for treating patients exhibiting the allergic reactions associated with seasonal allergic rhinitis, perennial allergic rhinitis chronic idiopathic urticaria and other similar allergic conditions.

### BACKGROUND OF THE INVENTION

Allergic diseases like allergic rhinitis, chronic idiopathic urticaria, asthma etc affect millions of people every year. Allergic rhinitis is of two types: seasonal allergic rhinitis and perennial allergic rhinitis. Seasonal allergic rhinitis is seasonal and is usually caused by pollen or mold, while perennial allergic rhinitis tends to be present for more than nine months of the year and can be attributed to dust mites, mold, animal dander or pollen in areas where high pollen counts are present for much of the year.

Chronic idiopathic urticaria is defined as the occurrence of wheals for duration of at least 6 weeks and is estimated to occur in 0.1 to 3% of the population. Its primary manifestation is smooth, edematous wheals surrounded by red flare. The presence of wheals is accompanied by intense itching and is associated with high morbidity.

Histamine plays an important role in the pathophysiology of allergic diseases. This mediator is found in its highest concentration in lungs, mast cells and basophils. Exposure to a sensitizing allergen results in the attachment of IgE molecules to the cells causing release of histamine and other inflammatory mediators.

The action of histamine at the H-1 receptor produces the classic symptoms of an allergic response: pruritis, wheal and flare reactions of the skin; sneezing, nasal pruritus, rhinorrhoea, palatal pruritus, itchy, red and watery eyes and congestion in the nose. In more severe circumstances, mucous membranes of the ears and paranasal sinuses can be involved producing symptoms of ear fullness and popping, itchy throat and pressure in the area above the cheeks and forehead. Fatigue, weakness and malaise can also be present. Patients with allergic rhinitis may be limited in their ability to perform activities and often note disturbances in sleep, work performance, concentration and quality of life.

It is now considered crucial to optimally treat an individual with allergic rhinitis as chronic inflammation of the nasal mucosa and nasal obstruction, if left untreated, can lead to more serious conditions of the upper and lower airways including asthma, sinusitis, chronic otitis media with effusion, and nasal and sinus polyps.

Desloratadine is a selective, H₁-receptor antihistamine. It is the major orally active metabolite of loratadine and acts by selectively blocking histamine at the histamine H₁-receptor. In vitro it inhibits release of histamine from human mast cells. Mean peak plasma concentrations of desloratadine are reached within 3 hours. Neither food nor grapefruit juice has an effect on the bioavailability of desloratadine. It is metabolized to an active metabolite, 3-hydroxydesloratadine, which subsequently undergoes glucuronidation.

Desloratadine is, however, known to react to form a degradant, N-formyl desloaratdine in tablet formulations. The formation of this N-formyl derivative is enhanced in the presence of acidic excipients such as silicon dioxide.

In general, tablet formulations require the presence of glidants. Silicon dioxide is the most widely used glidant in pharmaceuticals. Its small particle size and large specific surface area give it desirable flow characteristics that can be exploited to improve the flow properties of dry powders used in the tabletting processes.

In order to avoid formulation of the N-formyl degradant prior art methods suggest addition of a pharmaceutically acceptable basic salt like calcium dibasic phosphate, preparation of a coated system, preparation of non-hygroscopic compositions or avoiding the use of reactive components to improve the shelf life of the desloratadine composition and prevent the degradation of desloratadine in the composition.

U.S. Pat. No. 6,100,274 discloses a pharmaceutical composition for oral administration comprising an antiallergic effective amount of desloratadine in a pharmaceutically acceptable carrier medium comprising a desloratadine protective amount of a pharmaceutically acceptable basic salt and at least one pharmaceutically acceptable disintegrant.

The formulation of U.S. Pat. No. 6,100,274 addresses the issue of the stability of desloratadine, which is sensitive to the effects of acidic excipients. The '274 patent teaches that it is therefore a requirement to use a pharmaceutically acceptable basic salt in the formulation while avoiding the use of any acidic excipients.

The pharmaceutical composition of US2002/123504 (the '504 application) comprises a therapeutically effective amount of desloratadine wherein the pharmaceutical composition is substantially free of reactive components like lactose and monosaccharide and disaccharides. The '504 application discloses instant release solid pharmaceutical dosage forms comprising an open matrix network carrying a therapeutically effective amount of desloratadine or a pharmaceutically acceptable salt thereof, wherein the open matrix network comprises a carrier which does not interact with desloratadine. The '504 application discloses a composition in which active pharmaceutical ingredient is first granulated with an inert excipient, the resulting granules are coated with an inert or non-reactive coating agent, and finally the resulting coated granules blended with other excipients, including the reactive excipients. The '504 application further discloses a non-hygroscopic pharmaceutical composition comprising desloratadine or a pharmaceutically acceptable salt thereof, lactose and one or more pharmaceutically acceptable inert excipients, wherein the composition is substantially free of unbound water.

Accordingly, the '504 application deals with the issue of the instability of desloratadine and teaches that desloratadine reacts with lactose, resulting in a brown product. The '504 application further discloses a pharmaceutical composition not comprising such reactive components. Furthermore, the '504 application discloses the preparation of a non-hygroscopic composition, a coated composition or a matrix network to enhance the stability of the drug and reduce its degradation. It is therefore a requirement in the '504 application either to prepare a composition not comprising lactose or other similar reactive components, or to prepare a non-hygroscopic, coated or matrix composition to prevent the reaction of the desloratadine with the excipients.

The present invention provides a stable formulation of desloratadine that can be prepared without addition of a basic salt while still using silicon dioxide as glidant. Accordingly, the present invention has eliminated the use of a basic salt in combination with the active pharmaceutical ingredient while still using acidic components. The present invention has eliminated the need to prepare a non-hygroscopic or a coated composition to provide a stable and bioavailable desloratidine composition.

### SUMMARY OF THE INVENTION

The present invention addresses the issue of the instability of desloratadine with certain excipients and provides a simple composition that doesn't require addition of a basic salt or the preparation of a coated or non-hygroscopic composition to prevent the discoloration and degradation of the drug in the presence of acidic components, but yet has desirable stability and bioavailability characteristics. Compositions of the present invention also possess good shelf life.

### DETAILED DESCRIPTION OF THE INVENTION

Desloratadine has a high degree of therapeutic utility as some of the second-generation antihistamines were removed from the market because of their ability to block cardiac potassium channels resulting in the prolongation of the QT_{c} interval and development of torsades de pointes. No episode of QT prolongation or torsades de pointes associated with desloratadine was reported, but its degradation in the presence of certain excipients is a major problem because it reacts to form a degradant N-formyl desloratadine in tablet formulations. The formation of the N-formyl degradant was enhanced in the presence of acidic excipients like silicon dioxide.

Degradation of desloratadine is a major issue that not only affects the stability of the drug but also affects the bioavailability apart from the discoloration and degradation of the molecule. When a drug like desloratadine, which is susceptible to the effect of acidic components, is manufactured somewhat different formulations are required to be prepared so that it not only protects the drug from the effect of acidic excipients in a formulation but also provides a good release rate resulting in good bioavailability.

Desloratadine compositions are required for the management of certain allergic diseases. The present inventors have found that under the conventional method of preparing pharmaceutical dosage forms, desloratadine is not stable and forms degradant, N-formyl desloratadine in tablet formulations. Desloratadine contains an amine group in its structure and that has been found to react with conventional diluents such as lactose, resulting in a brown product. The present inventors have found that the presence of several triglycerides of fatty acids, like hydrogenated cottonseed oil, reduces interactions of desloratadine with acidic excipients like silicon dioxide and other similar substances. The formulations of the present invention can be prepared by the conventional methods like wet granulation, direct compression and dry granulation.

The new preparations are stable to the effect of acidic components and prevent the discoloration and degradation of desloratadine while still providing bioavailability to marketed formulations. The compositions of the present invention were obtained by intimately mixing desloratadine with an inert carrier and possibly other excipients, followed by compressing the mixture into a tablet and finally coating with aqueous dispersions of opadry coating material.

For the purpose of the present invention, active pharmaceutical ingredient used to achieve desired tablet composition comprises 90-96% of form-I of desloratadine and 4-10% of form-II of desloratadine.

Desloratadine, microcrystalline cellulose, corn Starch, mannitol, and colloidal silicon dioxide were sifted and mixed. The blend (or mixture) was lubricated with hydrogenated cottonseed oil and compressed into tablets. The tablets were coated with an aqueous dispersion of opadry coating material.

The present invention provides an immediate release composition of desloratadine that effectively protects desloratadine while providing acceptable release and bioavailability.

The present invention provides an immediate release composition of desloratadine that is simple to manufacture and does not involve cost intensive methods of preparation.

The compositions of the present invention do not require the use of a pharmaceutically acceptable basic salt in combination with desloratadine and do not even require the preparation of a coated or a non-hygroscopic composition.

The present invention is therefore a simple and cost-effective solution to the stability and bloavailability problems of conventional desloratadine formulations. Interestingly, problems related to stability, discolouration and bioavailability of desloratadine are solved by the compositions of the present invention without the need of a pharmaceutically acceptable basic salt and without the use of a coated system or a non-hygroscopic composition.

Tablets prepared according to the present invention has eliminated the use of a pharmaceutically acceptable salt of basic compound and it also is not required to prepare a coated or a non-hygroscopic composition, while using acidic excipients in the composition. Those skilled in the art will appreciate that a stable and bioavailable formulation of desloratadine could be obtained even without adding a pharmaceutically acceptable basic salt or without even preparing a coated or non-hygroscopic or anhydrous composition.

While desloratadine is a preferred drug according to the present invention, other acid sensitive drugs are also included within the scope of the present invention.

The compositions of the present invention is comprise an inert carrier, like hydrogenated cottonseed oil, to prevent the interaction between the acid (or acid labile) sensitive drug (such as desloratadine) and acidic excipients. The inert carrier appears to act as a molecular barrier that limits interactions between the drug and the excipients.

Inert materials which are suitable for the present invention include, but are not limited to, hydrogenated cottonseed oil, hydrogenated soyabean oil and other hydrogenated vegetable oils-type-I.

It is contemplated within the scope of the invention that other inert materials like waxes and polyethylene glycols may also be used to prepare a molecular barrier.

“Immediate release” as defined herein refers to making an active ingredient available to the biological system of the host. An immediate release preparation according to the present invention is one that provides stability to the drug while not delaying release of the dug, thus providing good bioavailability.

“Acid-sensitive” as defined herein refers to a drug or any material that degrades in the presence of acidic excipients. An acid labile drug according to the present invention is one that degrades to an inactive derivative upon being exposed to an acidic environment.

“Inert carrier” according to the present invention is one that facilitates the creation of a molecular barrier and does not react with any other component of the compositions of the present invention. The inert carrier thus isolates the drug from acidic excipients thereby preventing interactions between drug (desloratadine) and the excipients, resulting in enhanced stability of the formulation.

“Therapeutically effective amount” according to the present invention is meant an amount of active pharmaceutical ingredient in the pharmaceutical compositions which is effective to beneficially treat histamine induced disorders. The term “therapeutically effective amount” as used herein indicates the amount of desloratadine required to be administered to a subject in need thereof, to have the desired therapeutic effect. In accordance with the present invention, desloratadine is preferably used in an amount ranging from about 0.1 mg to about 15 mg.

The immediate release compositions of the present invention employ certain other excipients that, although not essential for the present invention, are required for the formulation process as known to those skilled in art.

The compositions of the present invention typically include pharmaceutically acceptable excipients. It is well known that pharmaceutical excipients are routinely incorporated into solid dosage forms. This is done to ease the manufacturing process as well as to improve the performance of the dosage form. Common excipients include, but are not limited to diluents, lubricants, granulating aids, surfactants, pH adjusters, anti-adherents and glidants etc. Such excipients may be routinely used in the dosage forms of the present invention.

If the desired formulation is a tablet, it may include one or more glidants, such as stearic acid, palmitic acid, talc, polyethylene glycol, colloidal silicon dioxide, carnauba wax and the like, and mixtures thereof. Other conventional pharmaceutical ingredients which may optionally be present include, but are not limited to, preservatives, stabilizers, and FD &C colours, etc.

The composition of the present invention may also comprise one or more of the following binders; corn starch, pregelantinized starch, hydroxypropyl cellulose, hydroxypropylmethyl cellulose, gelatin, and hydroxy ethyl cellulose or other similar substances.

The composition of the present invention may also include one or more diluents such as microcrystalline cellulose, starch, pregelantinized starch, mannitol or other similar substances.

The composition of the present invention may also include one or more disintegrants, such as low substituted hydroxy propyl cellulose, sodium starch glycolate, crospovidone, croscarmellose sodium or other similar substances.

While this invention has been described in terms of specific embodiments, it should be understood that presented by way of illustration only and that the invention is not necessarily limited thereto. Modifications and variations within the spirit and scope of the claims that follow will be readily apparent from this disclosure as those skilled in the art will appreciate.

The following examples serve to illustrate the present invention without limiting it.

### EXAMPLES

### Example 1

The immediate release oral pharmaceutical composition of the present invention was prepared as per the formula given in the Table 1 below. The example explains the use of acidic excipient colloidal silicon dioxide as glidant and hydrogenated cotton seed oil as lubricant as well as protecting agent.

TABLE 1

Quantity

| | | mg per | |
|---|---|---|---|
| Sr. No | Ingredients | tablet | % w/w |
| 1 | Desloratadine | 5 | 4.85 |
| 2 | Microcrystalline | 61.3 | 59.51 |
| | cellulose(Avicel PH112) | | |
| 3 | Corn Starch(Purity 21A) | 10 | 9.71 |
| 4 | Mannitol(Pearlitol SD200) | 21 | 20.39 |
| 5 | Colloidal silicon | 1.5 | 1.46 |
| | dioxide(Aerosil 200) | | |
| 6 | Hydrogenated cottonseed | 1.2 | 1.17 |
| | oil(Sterotex NF) | | |
| 7 | Opadry coating material | 3 | 2.91 |
| 8 | Film coated tablet weight | 103 | 100 |

Desloratadine, microcrystalline cellulose, corn starch, mannitol, and colloidal silicon dioxide were sifted and mixed. The blend was lubricated with hydrogenated cottonseed oil and compressed into tablets. The tablets were coated with aqueous dispersion of opadry coating material.

The tablets obtained were subjected to dissolution testing in 500 ml of 0.1N HCl using USP apparatus 2 (Paddles) at 50 rpm at 37±0.5° C. The results are presented in Table 2 below.

TABLE 2

| Sr. No | Time (In minutes) | % Drug released |
|---|---|---|
| 1 | 10 | 79 |
| 2 | 30 | 91 |
| 3 | 60 | 96 |

The tablets were subjected for stability studies. Results of the stability study are shown below in Table 3.

TABLE 3

| | Storage condition: | | | |
|---|---|---|---|---|
| Sr. No | 40° C./75% RH | Initial | 1 Month | 1.5 Month |
| 1 | N-Formyl | 0.19 | 0.24 | 0.29 |
| | desloratadine | | | |
| | impurity (%) | | | |
| 2 | Total impurities (%) | 0.19 | 0.35 | 0.34 |
| 3 | Description | White to off | White to off | Whit to off |
| | | white tablet | white tablet | white tablet |

### Example 2

Table 4 shows the composition of desloratadine tablet according to the present invention.

TABLE 4

| | | Quantity(mg | |
|---|---|---|---|
| Sr. No | Ingredients | per tablet) | Quantity(% w/w) |
| 1 | Desloratadine | 5 | 4.85 |
| 2 | Microcrystalline | 82.3 | 79.90 |
| | cellulose(Avicel PH112) | | |
| 3 | Corn Starch(Purity 21A) | 10 | 9.71 |
| 4 | Colloidal silicon | 1.5 | 1.46 |
| | dioxide(Aerosil 200) | | |
| 5 | Hydrogenated cottonseed | 1.2 | 1.17 |
| | oil(Sterotex NF) | | |
| 6 | Opadry coating material | 3 | 2.91 |
| 7 | Film coated tablet weight | 103 | 100 |

Desloratadine, microcrystalline cellulose, corn starch and colloidal silicon dioxide were sifted and mixed. The blend was lubricated with sifted hydrogenated cottonseed oil and compressed into tablets. The tablets were coated with aqueous dispersions of opadry coating material.

The tablets obtained were subjected to dissolution testing in 500 ml of 0.1N HCl using USP apparatus 2 (Paddles) at 50 rpm at 37±0.5° C.

The results are shown below in Table 5.

TABLE 5

| Sr. No | Time(In minutes) | % Drug released |
|---|---|---|
| 1 | 10 | 91 |
| 2 | 30 | 101 |
| 3 | 60 | 101 |

The tablets were subjected to stability studies. Results of the stability study are shown below in Table 6.

TABLE 6

| | Storage Conditions: | | |
|---|---|---|---|
| Sr. No | 40° C./75% RH | Initial | 1 Month |
| 1 | N-Formyl | 0.09 | 0.09 |
| | Desloratadine | | |
| | impurity (%) | | |
| 2 | Total Impurities (%) | 0.09 | 0.17 |
| 3 | Description | Whit to off | White to off |
| | | white tablet | white tablet |

### Example 3

Table 7 shows the composition of desloratadine tablet according to the present invention.

TABLE 7

Quantity

| Sr. No | Ingredients | mg per tablet | % w/w |
|---|---|---|---|
| 1 | Desloratadine | 5 | 4.85 |
| 2 | Microcrystalline | 82.3 | 79.90 |
| | cellulose(Avicel PH112) | | |
| 3 | Pregelantinized | 10 | 9.71 |
| | Starch(Starch 1500 LM) | | |
| 4 | Colloidal silicon | 1.5 | 1.46 |
| | dioxide(Aerosil 200) | | |
| 5 | Hydrogenated cottonseed | 1.2 | 1.17 |
| | oil(Sterotex NF) | | |
| 6 | Opadry coating material | 3 | 2.91 |
| 7 | Film coated tablet weight | 103 | 100 |

Desloratadine, microcrystalline cellulose, pregelantinized starch and colloidal silicon dioxide were sifted and mixed. The blend was lubricated with sifted hydrogenated cottonseed oil and compressed into tablets. The tablets were coated with aqueous dispersions of opadry coating material.

The tablets obtained were subjected to dissolution testing in 500 ml of 0.1 N HCl using USP apparatus 2 (Paddles) at 50 rpm at 37±0.5° C.

The results are shown below in table 8.

TABLE 8

| Sr. No | Time(in minutes) | % Drug released |
|---|---|---|
| 1 | 10 | 96 |
| 2 | 30 | 100 |
| 3 | 60 | 100 |

The tablets were subjected to stability studies. Results of the stability study are shown below in table no 9.

TABLE 9

| | Storage Conditions: | | |
|---|---|---|---|
| Sr. No | 40° C./75% RH | Initial | 1 Month |
| 1 | N-Formyl | 0.08 | 0.08 |
| | Desloratadine | | |
| | impurity (%) | | |
| 2 | Total impurities (%) | 0.13 | 0.13 |
| 3 | Description | White to off | White to off |
| | | white tablet | white tablet |

### Example 4

Table 10 shows the composition of desloratadine tablet according to the present invention.

TABLE 10

Quantity

| | | mg per | |
|---|---|---|---|
| Sr. No | Ingredients | tablet | % w/w |
| 1 | Desloaratdine | 5 | 4.85 |
| 2 | Microcrystalline | 80.3 | 77.96 |
| | cellulose(Avicel PH101) | | |
| 3 | Corn Starch(Purity 21 A) | 10 | 9.71 |
| 4 | Hypromellose 6 cps | 2 | 1.94 |
| 5 | Purified water | QS | QS |
| 6 | Colloidal silicon | 1.5 | 1.46 |
| | dioxide(Aerosil 200) | | |
| 7 | Hydrogenated cotton seed | 1.2 | 1.17 |
| | oil(Sterotex NF) | | |
| 8 | Opadry coating material | 3 | 2.91 |
| 9 | Film coated tablet weight | 103 | 100 |

Desloratadine, microcrystalline cellulose, corn starch were sifted and mixed. A binder solution was prepared by dissolving hyperomellose in purified water. The dry mix was granulated with the binder solution. The wet granules were dried, sifted and milled. Sifted granules were lubricated with sifted colloidal silicon dioxide and hydrogenated cottonseed oil. The lubricated blend was compressed into tablets. The tablets were coated with an aqueous dispersion of opadry coating material.

The tablets obtained were subjected to dissolution testing in 500 ml of 0.1 N HCl using USP apparatus 2(Paddles) at 50 rpm at 37±0.5° C.

The results are shown below in Table 11

TABLE 11

| Sr. No | Time(in minutes) | % Drug released |
|---|---|---|
| 1 | 10 | 80 |
| 2 | 30 | 92 |
| 3 | 60 | 102 |

### Example 5

Table 12 shows the composition of desloratadine tablet according to the present invention.

TABLE 12

| | | | |
|---|---|---|---|
| | | Quantity(mg | |
| Sr. No | Ingredients | per tablet) | Quantity (% w/w) |

Inner Phase:

| | | | |
|---|---|---|---|
| 1 | Desloratadine | 5.0 | 4.85 |
| 2 | Microcrystalline | 80.3 | 77.96 |
| | cellulose(Avicel | | |
| | PH101) | | |
| 3 | Corn Starch(Purity 21 A) | 10 | 9.71 |
| 4 | Hyperomellose 6 cps | 2 | 1.94 |
| 5 | Purified Water | QS | QS |

Outer Phase

| 6 | Colloidal Silicon | 1.5 | 1.46 |
|---|---|---|---|
| | Dioxide(Aerosil 200) | | |
| 7 | Hydrogenated | 1.2 | 1.17 |
| | cotton seed | | |
| | oil(Sterotex NF) | | |
| Core Tablet Weight | | 100 | 97.09 |
| 8 | Opadry coating | 3 | 2.91 |
| | material | | |

| | | |
|---|---|---|
| Film coated tablet weight | 103 | 100 |

Desloratadine, microcrystalline cellulose, corn starch were sifted and mixed. A binder solution was prepared by dissolving hyperomellose in purified water. The dry mix was granulated with the binder solution. The wet granules were dried, sifted and milled. Sifted granules were lubricated with sifted colloidal silicon dioxide and hydrogenated cottonseed oil. The lubricated blend was compressed into tablets. The tablets were coated with an aqueous dispersion of opadry coating material.

The tablets obtained were subjected to dissolution testing in 500 ml of 0.1 N HC lusing USP apparatus 2(Paddles) at 50 rpm at 37±0.5° C.

The results are shown below in table 13.

TABLE 13

| Sr. No | Time(in minutes) | % Drug Released |
|---|---|---|
| 1 | 10 | 80 |
| 2 | 30 | 92 |
| 3 | 60 | 102 |

The tablets were subjected to stability studies. Results of the stability study are shown below in table 14.

TABLE 14

| | Storage Conditions: | | |
|---|---|---|---|
| Sr. No | 40% C./75% RH | Initial | 1 Month |
| 1 | N-Formyl | 0.11 | 0.11 |
| | desloratadine | | |
| | impurity (%) | | |
| 2 | Total impurities (%) | 0.12 | 0.18 |
| 3 | Description | White to off | White to off |
| | | white tablet | white tablet |

### Example 6

Table 15 shows the composition of desloratadine tablet according to the present invention.

TABLE 15

| | | | |
|---|---|---|---|
| | | Quantity(in mg | Quantity(in % |
| Sr. No | Ingredients | per tablet) | w/w) |

Inner Phase

| | | | |
|---|---|---|---|
| 1 | Microcrystalline | 77.3 | 75.05 |
| | Cellulose(Avicel | | |
| | PH101) | | |
| 2 | Pregelantinized | 15.0 | 14.56 |
| | Starch(Starch 1500) | | |
| 3 | Colloidal Silicon | 1.5 | 1.46 |
| | dioxide(Aerosil 200) | | |

Outer Phase

| | | | |
|---|---|---|---|
| 4 | Desloratadine | 5.0 | 4.85 |
| 5 | Hydrogenated cottonseed | 1.2 | 1.17 |
| | oil(Sterotex NF) | | |

| | | |
|---|---|---|
| Core Tablet Weight | 100 | 97.09 |

Film Coating

| | | | |
|---|---|---|---|
| 6 | Opadry Coating Material | 3 | 2.91 |

| | | |
|---|---|---|
| Film Coated Tablet Weight | 103 | 100 |

Microcrystalline cellulose, pregelantinized starch and colloidal Silicon dioxide were sifted and mixed. The dry mix was granulated with purified water. The wet granules were dried, sifted and milled. Sifted granules were blended with sifted desloratadine and lubricated with sifted hydrogenated cottonseed oil. The lubricated blend was compressed into tablets. The tablets were coated with an aqueous dispersion of opadry coating material.

The tablets obtained were subjected to dissolution testing in 500 ml of 0.1N HCl using USP apparatus 2 (Paddles) at 50 rpm at 37±0.5° C.

The results are shown below in Table 16.

TABLE 16

| Sr. No | Time(In minutes) | % Drug Released |
|---|---|---|
| 1 | 10 | 79 |
| 2 | 30 | 94 |
| 3 | 60 | 98 |

The tablets were subjected for stability studies. Results of stability study are shown below in table 17.

TABLE 17

| | Storage Condition | | | |
|---|---|---|---|---|
| Sr. No | 40% C./75% RH | Initial | 1 Month | 2 Month |
| 1 | N-Formyl | 0.16 | 0.21 | 0.27 |
| | Desloratadine | | | |
| | impurity (%) | | | |
| 2 | Total impurities (%) | 0.16 | 0.21 | 0.27 |
| 3 | Description | White to off | White to off | White to off |
| | | white tablet | white tablet | white tablet |

1. An active pharmaceutical ingredient comprising 90-96% of form I of desloratadine and 4-10% of form 11 of desloratadine.

2. The active pharmaceutical ingredient of claim 1 wherein form I of desloratadine is present in an amount ranging from 93-95% and form 11 of desloratadine is present in an amount ranging from 5-7%.

3. The active pharmaceutical ingredient of claim 1 comprising 95% of form I of desloratadine and 5% of form 11 of desloratadine.

4. A process for preparing an active pharmaceutical ingredient comprising 90-96% of form I of desloratadine and 4-10% of form II of desloratadin, the method comprising intimately mixing the form I of desloratadine with form II of desloratadine.

5. An immediate release pharmaceutical composition comprising 90-96% of form I of desloratadine or a pharmaceutically acceptable salt thereof and 4-10% of form II of desloratadine or a pharmaceutically acceptable salt thereof, and at least one pharmaceutically acceptable excipient.

6. The pharmaceutical composition of claim 5 comprising 93-95% of form I of desloratadine or a pharmaceutically acceptable salt thereof and 5-7% of form II of desloratadine or a pharmaceutically acceptable salt thereof, and at least one pharmaceutically acceptable excipient.

7. The pharmaceutical composition of claim 5 comprising 95% of form I of desloratadine or a pharmaceutically acceptable salt thereof and 5% of form II of desloratadine or a pharmaceutically acceptable salt thereof, and at least one pharmaceutically acceptable excipient.

8. The pharmaceutical composition of claim 5 wherein a therapeutically effective amount of desloratadine is 2.5 to 5 mg.

9. The pharmaceutical composition of claim 5 wherein a therapeutically effective amount of desloratadine is about 5 mg.

10. The pharmaceutical composition of claim 5 further comprising at least one acidic excipient.

11. The pharmaceutical composition of claim 5 wherein said pharmaceutically acceptable excipient is hydrogenated vegetable oil type-I, a wax or polyethylene glycol.

12. The pharmaceutical composition of claim 5 which is in the form of a capsule or a tablet.

13. The pharmaceutical composition of claim 12 which is in the form of a tablet.

14. A pharmaceutical composition which prevents degradation of desloratadine in the presence of acidic excipients comprising a therapeutically effective amount of desloratadine or a pharmaceutically acceptable salt thereof, and at least one pharmaceutically acceptable excipient.

15. The pharmaceutical composition of claim 14 wherein said composition has a pH of about 3 to about 7.

16. The pharmaceutical composition of claim 14 wherein said composition has a pH of about 3 to about 5.

17. The pharmaceutical composition of claim 14 wherein said acidic excipient is colloidal silicon dioxide, sodium starch glycolate type B or low-substituted hydroxyl propyl cellulose.
